(19) United States
(12) Patent Application Publication
(10) Pub. No.: US 2006/0257282 A1
(43) Pub. Date: Nov. 16, 2006

(54) Large-scale decontamination of biological microbes using amine oxides at acidic pH

(76) Inventors: Tony Buhr, King George, VA (US); Andrew Slaterbeck, King George, VA (US)

Correspondence Address: NAVAL SURFACE WARFARE CENTER, DAHLGREN DIVISION;OFFICE OF COUNSEL, CODE XDC1, 17320 DAHLGREN ROAD, DAHLGREN, VA, 22448-5110, US

(21) Appl. No.: 11/129,753

(22) Filed: May 12, 2005

(51) Int. Cl. A61L 2/18

(52) U.S. Cl. 422/28

(57) ABSTRACT

A large area decontaminating method for biological spore populations uses the application of an acidic environment in combination with an amine oxide to decontaminate the spores.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefore.

### FIELD OF THE INVENTION

The present invention provides a method for large-scale decontamination of biological microbes using amine oxides at acidic pH.

### BACKGROUND OF THE INVENTION

There are numerous strategies for biological decontamination. These include both general and specific decontamination strategies. General decontamination strategies include using strong acids, strong bases, strong oxidizers such as Clorox (hypochlorite), or gases such as methyl bromide. The problem with these strategies is that they are environmentally unfriendly, highly toxic, and/or corrosive, which limits their applications. When used in decontamination processes, e.g., processes generally used over large areas of coverage, strong chemicals become problematic as environmentally toxic and hazardous to people. In particular, these options could not be used to decontaminate in situations that necessitated contact of the chemicals with human skin. Specific decontamination strategies would include utilizing enzymes or developing biologically based solutions. The difficulty with specific decontaminants are limited efficacy, especially over a wide temperature range or a short time frame, high costs, limited availability, short shelf lives, and problems related to the decontaminant being readily inactivated.

There is a need in the art to provide an effective and safe methodology for large scale decontamination. The present invention addresses this and other needs.

### SUMMARY

The present invention includes a method for large-scale decontamination of biological microbes consisting essentially of the steps of applying an acidic environment to large-scale biological spores contamination and applying an amine oxide compound in combination with the acidic environment effective for large-scale decontamination of the biological microbes. In one preferred embodiment, the decontamination occurs at an elevated temperature environment. The present invention also includes a large-scale decontaminated microbe product produced by the above-described method.

The application of an amine oxide in an acidic environment for use in large-scale biological decontamination provides an effective methodology while not posing the environmental hazards or human toxicity levels associated with harsh chemicals such as concentrated hypochlorite or methyl bromide.

The present invention also include a method for large-scale decontamination of biological microbes comprising the steps of washing a biological microbe contaminant from a decontamination site into a waste water product, applying an acidic environment to the contaminated waste water product and applying an amine oxide compound in combination with the acidic environment effective for decontamination of the biological microbes within the waste water product.

The present invention provides solutions to decontaminate biological organisms including bacterial endospores, particularly spores produced by Bacillus species. Decontamination (decon) is the process of killing, removing and/or inhibiting (neutralizing) any harmful agents including biological organisms and/or chemicals.

### BRIEF DESCRIPTION OF THE DRAWINGS

FIG. 1 is a graph showing decontamination of B. globigii spores with 1% isoalkyl dimethyl amine oxide at 25° C. and 70° C.;

FIG. 2 is a graph showing decontamination of B. globigii spores with 1% cocoa dimethyl amine oxide at 25° C. and 70° C.; and,

FIG. 3 is a graph showing decontamination of B. globigii spores with 1% decyl dimethyl amine oxide at 25° C. and 70° C.

### DETAILED DESCRIPTION

The present invention provides a method for decontaminating biological spores using the application of an amine oxide in an acidic environment. The method of the present invention includes large-scale decontamination of biological microbes by applying an acidic environment to large-scale biological spores contamination, and within that acidic environment, applying an amine oxide compound onto the contaminant microbes in a manner for effective large-scale decontamination of the biological microbes. The application of the present invention to the microbe populations also may include heated conditions, preferably under moderately heated conditions. The method of the present invention decontaminates the spores with minimal environmental or health risks while being effective for decontaminating biological microbes, such as endospores within the Bacillus endospores family, including B. globigii spores. This allows effective and rapid decontamination of large areas permitting ready use of these large areas, such as for military or civilian operations.

The amine oxide of the present invention has the general formula of:
R₁R₂R₃N→O;

with R₁ being a substituted or unsubstituted alkyl or alkenyl group containing from about 5 to about 25 carbon atoms, such as about 8 to about 20 carbon atoms. R₂ and R₃, each, being a substituted or unsubstituted alkyl or alkenyl groups containing from about 1 to about 15 carbon atoms, such as from about 1 to about 5 carbon atoms. Preferably, the amine oxide of the present invention includes a dimethyl amine oxide, such as decyl dimethyl amine oxide, cocoa dimethyl amine oxide, isoalkyl dimethyl amine oxide or combinations thereof. Aqueous amine oxide formulations for use in the present invention may be prepared by known and conventional methods, such as the controlled oxidation of tertiary amines to the corresponding amine oxide using a strong oxidizing agent. One such known oxidizing agent is hydrogen peroxide. With the addition of hydrogen peroxide solution, in a stochiometric or greater amount, to an aqueous solution of the tertiary amine, the derivative amine oxide is formed. Methods for producing the amine oxide are described in U.S. Pat. Nos. 3,215,741; 3,223,647 and 4,565,891, the disclosure of which are herein incorporated by reference. Additionally, amine oxides are commercially available, such as from Lonza of Basel, Switzerland. The type and amount of amine oxide useful for decontamination of a particular spore population may be determined by those skilled in the art of decontamination in light of the disclosure herein. Relevant factors useful in determining the most appropriate heated acidic conditions to be used include such items as the type of spore species, type of surface or area to be decontaminated, amount of contamination, environmental conditions of the cleanup, and other such criteria that are determinable by those skilled in the art of decontamination.

An acidic pH environment appears critical for sporicidal activity. Application of the acidic environment includes any appropriate methodology for exposing the spores, such as by suspending, surrounding, encasing, inundating, engulfing, submerging, misting, or otherwise subjecting the spores to the acidic environment as to affect the spores thereby. Methodologies may include sprays, mists, liquids, solids and the like. Preferably application of the acidic environment comprises application of an acidic solution to the spores or to an environment to which the spores are introduced. Acidic environments include, for example, pH ranges of less than about 6.9, preferably from about 1.0 to less than about 6.9, more preferably from about 3.0 to about 6.0, and still more preferably from about 4.0 to about 6.0, and most preferably about 5.0. Acidic solutions may include carboxylic acids, organic acids, inorganic acids, and combinations thereof, including such acids as oxalic acid, acetic acid, phosphoric acid, hydrochloric acid, citric acid, sulfuric acid, nitric acids, and the like. In solution the acids may be present in amounts that allow the convenient and non-hazardous application onto the spores. Such amounts may include, for example, from about 1 mM to about 1,000 mM, from about 2 mM to about 500 mM, from about 10 mM to about 100 mM, from about 20 mM to about 50 mM, and the like. When solutions are applied to a spore population, preferably enough solution is applied to completely immerse the spores in the solution. Application of the acidic environment may include application of an acidic solution in a moderately heated condition. Most preferably, the acid solution is selected for specific locations and uses to minimize harmful effects, such as harmful effects on humans or equipment.

In one preferred embodiment referred to above, the amine oxide in combination with the acidic environment is applied in a heated condition, such as a moderately heated condition. The moderately heated condition of the spores includes above-ambient temperatures that in combination with the acidic environment decontaminate the spore population. Representative temperatures of a heated condition include, for example, non-destructive temperatures for a given object to be decontaminated, such as temperatures below the boiling point of an aqueous constituent of the object to be decontaminated or any constituent part of the acidic environment, e.g., from about 100° C. or less, from about 25° C. to about 100° C., from about 50° C. to about 100° C., from about 65° C. to about 85° C., from about 75° C. to about 80° C., and the like. Preferred temperatures include, for example without limitation, heating the biological spore environment to a temperature of from about 25° C. to about 70° C., or a more specific temperature of about 70° C. Methods of heating may include imparting heat into the acidic environment/spore population including, for example, use of exothermic chemical reaction, use of external heat source such as heating elements, combinations of these methods, and the like.

Methodologies of the decontamination of the spores include the spores being subjected to the acidic environment and amine oxide, and simultaneously or sequentially, exposed to a heated condition. Heat may be applied prior to, during or after the application of the acid environment and/or amine oxide. Acidic and heated conditions may be varied by the type and amount of spore population, and by the object or environment to be decontaminated. In one preferred embodiment, the biological spores are collected from a contaminated area and placed in a container for application of dilute acid and heat, such as in the form of a heating element. Alternatively, the application of preheated dilute acid is used for decontamination on site.

In one application of the present invention, spores may be harvested and/or trapped by vacuums, filters, glues, etc. to collect and concentrate the spores and placed into a container or similar retaining device. Within the container, the spores are exposed to appropriate amine oxide/acidic conditions, which may be present when the spores were placed in the container or added after placement of the spores, and heated with a heating element for an appropriate time period to kill and decontaminate the spores. Such decontamination devices may be small, inexpensive and readily transportable, having an acid resistant container, such as a glass-based, resilient plastic or stainless steel composition, and a heating component such as a heating element. In a second embodiment, an exothermic chemical reaction may be used in place of the heating element for imparting heat to the amine oxide/acidic environment to kill spores in contaminated areas. Numerous factors, determinable by artisans in the decontamination arts, affect the efficiency of the decontamination method of the present invention. Such factors include, for example, the selection of acid or amine oxide, amine oxide or acid concentration, uniformity of heat, variations in heat temperatures, time period of application, type of spore, etc., with the optimum decontamination conditions determinable by those skilled in the art through routine experimentation in light of the disclosure herein. Generally, selection of appropriate decontamination procedures includes a balance of acid strength, heat conditions, time constraints (such as operational military timetables), environmental sensitivities to acidic and/or elevated heated conditions, and toxicity for a given spore hazard. Preferably decontamination occurs within about 60 minutes, such as 15 minutes, 30 minutes or 45 minutes.

The large-scale decontaminated microbe population produced by the present invention overcomes safety issues, particularly health problems, for the effective use of decontamination methods. Additionally, the present invention addresses on-going use of contaminated equipment, such as military aircraft, ships and the like, after decontamination of this equipment. The use of large amounts of an amine oxide in an acidic environment has been found effective for decontamination without significant health risks associated with other decontaminant procedures.

Bacterial endospores are one of the most difficult biological agents to kill and decontaminate. Success with decontamination of bacterial spores is a strong benchmark for the solutions to be effective over many types and forms of microbes. The amine oxide acidic solutions are not exclusive to decontamination of Bacillus endospores and can be used for spores of other bacteria including Clostridium, or other forms of bacteria including vegetative bacteria and other microbes including other species of bacteria, viruses and fungi. The present invention is particularly applicable for decontamination of biological spores, particularly biological spores that comprise bacterial endospores. As used herein, the terms spores, biological spores, spore populations and similar terminology, refer to contaminant spores that create a hazard, threat, nuisance, etc. by their presence in an environment, on a surface, in food, etc. Typical spores decontaminated by the present invention include, for example, endospores, such as those belonging to the genus Bacillus, Clostridium, and the like. Representative endospore populations include Bacillus and Clostridium species. Some examples are Bacillus cereus, Bacillus anthracis and Bacillus globigii.

Effectiveness of the methodology of the present invention occurs with increases of biological spore kills with the use of the amine oxide/acidic environment, optionally with heat, over the non-use of such conditions. Preferably, an effective kill is variable, depending on the original number of spores within a contamination, such as a 90% effectiveness (kill) against a concentration of 103 spores/ml, and more preferably an effectiveness of 90% against a concentration of 108 spores/ml, with a most preferred decontamination of from about six or more logarithmic reductions of live spores. Most preferably, the decontamination reduces the spore concentration to a level that renders the once hazardous contaminated area or surface no longer hazardous. Effective biological spore decontamination of these spores rids a contaminated space or object of the immediate hazard occasioned by the spore presence. Spores are killed when they are rendered harmless, i.e., no longer hazardous, to a living organism, particularly a human. Depending on the circumstances, spore decontamination may be desirable against spores that affect other mammals, animals or plants. Decontamination applications non-exclusively include decontamination of endospore-forming bacteria in military, medical, industry, agriculture, and household domains, particularly in the event of accidental contamination or terrorist attack. Representative localities that could benefit from the decontamination regimen of the present invention for reduction of spore populations include hospitals, veterinary clinics, farms, dairies, meat processing facilities, hide processing facilities, ships, buildings, houses, automobiles, and other like contaminated surfaces and/or areas.

Advantageously the present invention provides low toxicity, low cost, high availability of acids and reduced logistical problems for decontamination. Selection of toxicity, kill ratios for a given spore population, and heat conditions may be varied to treat specific contaminated environments or surfaces. Within working ranges of the present invention, such as typical amounts of 10 mM or 100 mM, acids are dilute, inexpensive and readily available. Logistical concerns are mitigated because acids can be transported in small volumes of concentrated amounts and diluted prior to use. These dilute acids of the present invention have minimal toxicities for decontamination of a given hazard, and are much more environmentally friendly than the same acids in more concentrated amounts.

### Methodology

In the examples, below, three representative amine oxides were tested for decontamination of Bacillus spores. The three exemplified amine oxides are isoalkyl dimethyl amine oxide, lauryl (cocoa) dimethyl amine oxide and decyl dimethyl amine oxide. All three amine oxides were dissolved in water and tested over a pH range. Testing occurred with and without applied heat. All experiments were performed with at least 3-4 replicates with many experiments reproduced numerous times.

Bacillus spores were suspended in the appropriate solvent at a concentration of 2 mg spores/ml (approximately 2×10⁸ spores/ml). Starting volume of 1.125 ml of spores at 2 mg/ml. 3 or 4 spore aliquots of 50 ul per aliquot were removed. These aliquots were serially diluted in 1×PBS and plated on LB or TSA agar plates. This is a control step that gave the spore titer or colony-forming units (CFUs) at Time=0 minutes (T0). The appropriate decontamination solution (2× concentration) was aliquoted into each of 3 or 4 Eppendorf tubes with 200 ul per tube. Then 200 ul of spores were added and mixed to give a final concentration of 1× decontamination solution and 1 mg spores/ml. The spores in decontamination solution were then mixed and incubated for specific time periods and temperatures. A typical incubation was in a room temperature (25° C.) water bath for 30 (or 60) minutes. There was no agitation during the incubation period in order to mimic real world situations. Aliquots of spores were removed after 30 (or 60) minutes. The aliquots (3 or 4 replicates) were serially diluted in 1×PBS and plated on LB or TSA agar plates. This gave a CFU at Time=30 minutes (T30), which is the titer of live spores after the decontamination test. After removing the aliquots at the 30-minute time point, test (or control) samples were then immediately incubated in a 70° C. water bath for another 30 minutes. The samples were then serially diluted and plated on to LB or TSA agar plates to determine the titer after the 70° C. incubation. The entire dilution series from 0.1 through 0.000001 dilutions were plated. An optional wash assay was employed to confirm that the surfactant was killing spores rather than behaving as a bacterostatic agent. The wash assay included centrifuging and washing spores after decontamination. 100 ul of spores (after decontamination) were added to 200 ul of water and centrifuged at 10,000×g for one minute at room temperature. The supernatant was removed and saved. 10% of the supernatant volume was plated and titered as a wash control, to determine if live spores were being removed in the supernatant. Spore pellets were suspended in 1 ml of water and then pelleted at 10,000×g for one minute at room temperature. The supernatant was removed and saved. Spore pellets were washed again with 1 ml of water. Then spore pellets were suspended, serially diluted and plated. This removed the surfactant prior to plating and confirmed results that were bactericidal rather than bacterostatic.

### EXAMPLE 1

### Spore Decontamination Using Isoalkyl Dimethyl Amine Oxide Suspended in Water at pH5 Through pH9 with or without Applied Heat

Isoalkyl dimethyl amine oxide, having a branched C₁₂ component, was dissolved in water. The isoalkyl dimethyl amine oxide was obtained from Lonza of Basel, Switzerland, manufactured under the trade name Barlox 12i. The isoalkyl dimethyl amine oxide was shipped as a 30% solution in water. Barlox 12i was either diluted in water without adjusting the pH, or samples were pH-adjusted using HCl or NaOH.

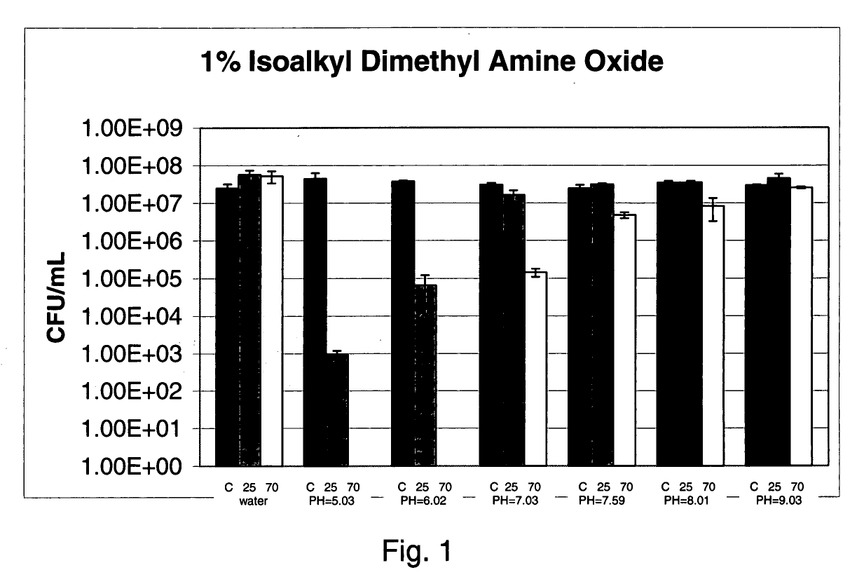

Referring to FIG. 1, decontamination of B. globigii spores is shown after spores were suspended in isoalkyl dimethyl amine oxide at a final concentration of 1%, and a spore concentration greater than 107 spores per milliliter for 30 minutes at 25° C., or for 30 minutes at 70° C. The assay used to generate FIG. 1 was decontamination followed immediately by dilution and plating. As seen in FIG. 1, spore decontamination after 30 minutes at 25° C. (room temperature) was strongly dependent on pH (“25” designated bars in FIG. 1). “C” designates the starting spore concentration. The amine oxide had no decontamination efficacy at basic pH, but efficacy was immediately enhanced as pH became acidic. There was greater than 2-orders-of-magnitude reduction at pH6 and greater than 4-orders-of-magnitude reduction at pH5. Decontamination remained strongly pH dependent at 70° C. (“70” designated bars in FIG. 1). There was some decontamination at neutral pH7, but complete decontamination occurred at acidic pH5-6. Moderate heat (70° C.) increased the efficacy of decontamination compared to 25° C.

An optional wash assay was employed to verify the results (data not shown). The purpose of the wash assay was to remove the decontaminant (amine oxide surfactant) prior to plating to reduce the likelihood that the amine oxide was binding to spores and simply inhibiting spore growth, i.e., this assay confirmed there were no false positives in the previous assay. The wash assay showed nearly identical spore decontamination between pH5 and pH7 at 70° C. as the dilution plating assay. Thus, results from both assays agreed for acidic to neutral pH between pH5 and pH7.

### EXAMPLE 2

### Spore Decontamination Using Cocoa Dimethyl Amine Oxide Suspended in Water at pH4 Through pH9 with or without Applied Heat

Cocoa dimethyl amine oxide (C₁₂, C₁₄, C₁₆) was dissolved in water. Lonza manufactures the amine oxide surfactant under the trade name Barlox 12. It is shipped as a 30% solution in water. Barlox 12 was either diluted in water without adjusting the pH, or samples were pH-adjusted using HCl or NaOH.

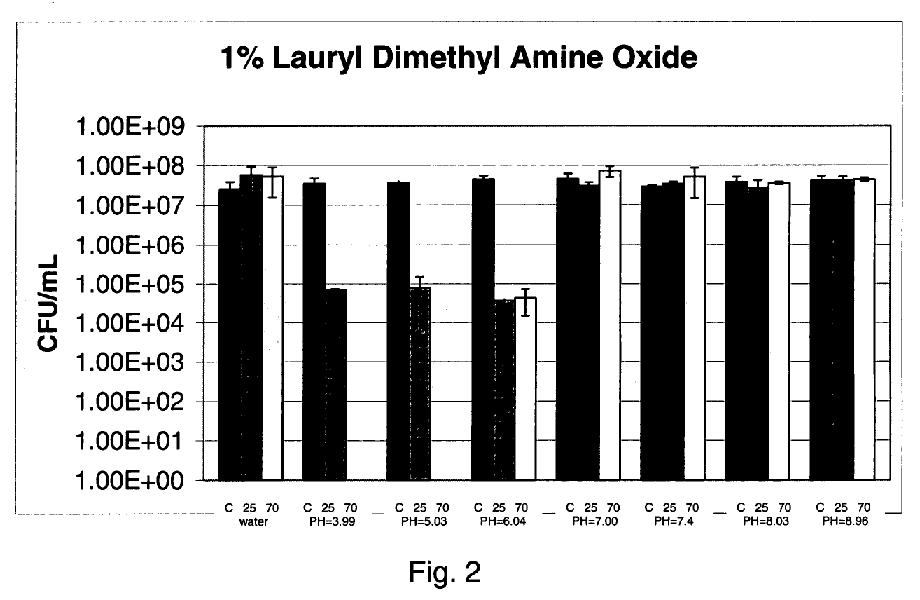

Referring to FIG. 2, decontamination of B. globigii spores is shown after spores were suspended in cocoa dimethyl amine oxide at a final concentration of 1%, and a spore concentration greater than 107 spores per milliliter for 30 minutes at 25° C., or for 30 minutes at 70° C. The assay used to generate FIG. 2 was decontamination followed immediately by dilution and plating. Spore decontamination after 30 minutes at 25° C. (room temperature) was strongly dependent on pH (“25” designated bars in FIG. 2). “C” designates the starting spore concentration. The amine oxide surfactant had no decontamination efficacy at basic pH, but efficacy was immediately enhanced as pH became acidic. There was greater than 2-orders-of-magnitude reduction at pH4-6 at room temperature. Decontamination remained strongly pH-dependent at 70° C. (“70” designated bars in FIG. 2). There was no decontamination at neutral pH7 or at basic pH. There was greater than 2-orders-of-magnitude decontamination at pH6, but complete decontamination did not occur until pH5. Moderate heat (70° C.) increased the efficacy of decontamination compared to 25° C.

### EXAMPLE 3

### Spore Decontamination Using Decyl Dimethyl Amine Oxide Suspended in Water at pH4 Through pH9 with or without Applied Heat

Decyl dimethyl amine oxide was dissolved in water. Lonza manufactures the amine oxide under the trade name Barlox 10S. It is shipped as a 30% solution in water. Barlox 10S was either diluted in water without adjusting the pH, or samples were pH-adjusted using HCl or NaOH.

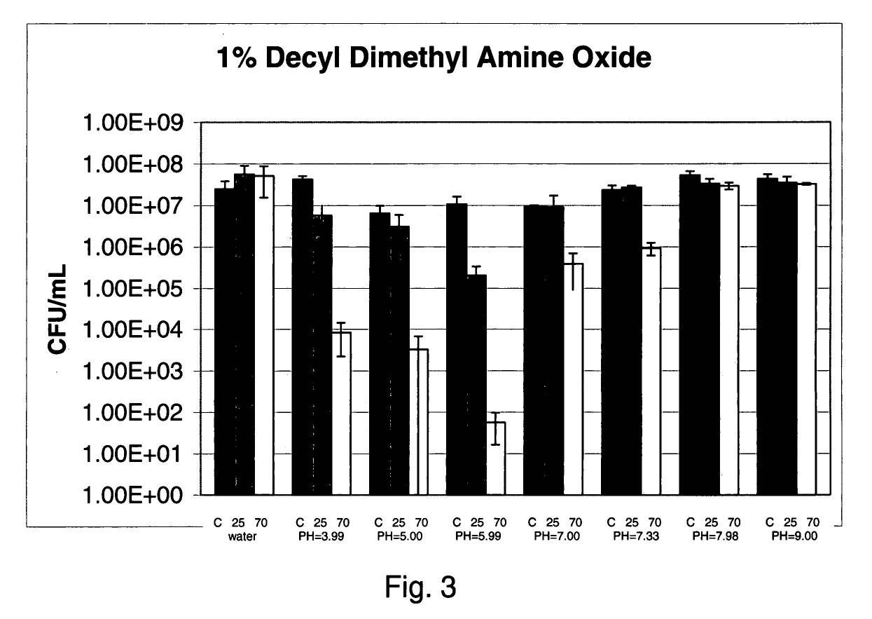

Referring to FIG. 3, decontamination of B. globigii spores is shown after spores were dissolved in decyl dimethyl amine oxide at a final concentration of 1%, and a spore concentration greater than 107 spores per milliliter for 30 minutes at 25° C., or for 30 minutes at 70° C. The assay used to generate FIG. 3 was decontamination followed immediately by dilution and plating. Spore decontamination after 30 minutes at 25EC (room temperature) was dependent on pH (“25” designated bars in FIG. 3). “C” designates the starting spore concentration. The amine oxide had no decontamination efficacy at basic pH, but efficacy was immediately enhanced as pH became acidic. Efficacy was not as strong compared to the previous amine oxides. pH6 appeared to be the optimum pH within the pH range that was tested. Decontamination was strongly pH dependent at 70° C. (“70” designated bars in FIG. 3). There was little or no decontamination at neutral pH7 or at basic pH. There was greater than 5-orders-of-magnitude decontamination at pH6, and pH6 showed the highest efficacy. There was significant sporicidal activity at pH4-5, but it wasn't quite as good as pH6. Furthermore, the efficacy of this surfactant was less than the other amine oxides. The Barlox 10S tail is 2 carbons shorter compared to the other amine oxides. Thus, pH and tail length apparently have significant impacts on decontamination efficacy for the amine oxides. Moderate heat (70° C.) increased the efficacy of decontamination compared to 25° C.

The three amine oxide surfactants (isoalkyl dimethyl amine oxide, cocoa dimethyl amine oxide, decyl dimethyl amine oxide) tested were each shown to have strong sporicidal (bacterial) activity in the presence of moderate applied heat. Isoalkyl dimethyl amine oxide showed strongest sporicidal efficacy followed closely by lauryl dimethyl amine oxide and finally decyl dimethyl amine oxide. Decontamination efficiency was strongly dependent on pH, and acidic pH was a requirement. The amine oxide surfactants have moderate to strong sporicidal activity at acidic pH, and activity is increased when combined with moderate heat.

### EXAMPLE 4

### Prophetic

Decontamination teams arrive at a contaminated area and set up an operations tent for decontamination. The teams conduct a large scale wash-down of individuals and equipment, and collect the used water (“gray water”) in holding containers. The gray water is acidified and an amine oxide is added. The holding containers are further heated with external heating elements. The gray water is held for one hour 70° C. in the holding containers and then discarded. The holding containers are used again for decontaminating another batch of contaminated waste water.

### EXAMPLE 5

### Prophetic

Decontamination teams arrive at a contaminated area and set up an operations tent for decontamination. The teams conduct a large scale wash-down of individuals and equipment using an amine oxide solution at pH5-pH6, and collect the used wash solution (“gray washdown”) in holding containers. The gray washdown in the holding containers is heated by circulating the gray water through a radiator from an internal combustion engine, which are typically maintained at 90° C.-95° C. The average temperature of the gray washdown in the holding tank is maintained at 70° C. or higher for 30 minutes or longer by circulating the large volume of water through the radiator. The decontaminated gray washdown could then be recycled or discarded. The holding containers are used again for decontaminating another batch of contaminated gray washdown.

Decontamination applications for the present invention include military, medical, industry, agriculture, household areas, such as from accidental contamination or in the event of terrorist attacks. The decontamination solutions are useful for reducing microbe populations in hospitals, veterinary clinics, farms, dairies, meat processing facilities, hide processing facilities, ships, buildings, houses, automobiles, and other various contaminated surface and/or area, including for example skin, hair and clothes. The efficacy against bacterial spores is a strong indicator that amine oxide surfactants used at acidic pH, plus moderate heat will be effective against a wide range of biological agents because bacterial spores are considered one of the most difficult to decontaminate. Effectiveness of the amine oxide acidic solution may include removal of many biological agents, including Bacillus endospores and other bacteria including Clostridium, or nonspore-forming bacteria such as vegetative bacteria and other microbes including fungi, viruses and possibly toxins. The amine oxide surfactants are generally inexpensive, highly available and have a long shelf life.

The foregoing summary, description, and examples of the present invention are not intended to be limiting, but are only exemplary of the inventive features which are defined in the claims.

What is claimed is:

1. A method for large-scale decontamination of biological microbes, consisting essentially of the steps of:
applying an acidic environment to large-scale biological spores contamination; and,
applying an amine oxide compound in combination with the acidic environment effective for large-scale decontamination of the biological microbes.

2. The method of claim 1, wherein the step of applying an acidic environment comprises application of an acidic solution.

3. The method of claim 1, wherein the step of applying an acidic environment comprises a pH of less than about 6.9.

4. The method of claim 3, wherein the step of applying an acidic environment comprises a pH of from about 3.0 to about 6.0.

5. The method of claim 4, wherein the step of applying an acidic environment comprises a pH of from about 4.0 to about 6.0.

6. The method of claim 2, wherein the acidic solution is selected from the group consisting of organic acids, inorganic acids and combinations thereof.

7. The method of claim 2, wherein the acidic solution is selected from the group consisting of oxalic acid, acetic acid, phosphoric acid, hydrochloric acid, sulfuric acid, carboxylic acids, and combinations thereof.

8. The method of claim 2, wherein the acidic solution is present in an amount of from about 1 mM to about 1,000 mM.

9. The method of claim 8, wherein the acidic solution is present in an amount of from about 20 mM to about 50 mM.

10. The method of claim 1, wherein the amine oxide is selected from the group consisting of decyl dimethyl amine oxide, cocoa dimethyl amine oxide, isoalkyl dimethyl amine oxide and combinations thereof.

11. The method of claim 1, further comprising the step of placing the applied acidic environment in a heated condition.

12. The method of claim 11, wherein the heated condition includes a moderately heated condition.

13. The method of claim 12, wherein the step of moderately heating the biological spores comprises a temperature of from about 25° C. to about 100° C.

14. The method of claim 13, wherein the step of moderately heating the biological spores comprises a temperature of from about 65° C. to about 85° C.

15. The method of claim 1, wherein the step of moderately heating the biological spores comprises an exothermic chemical reaction.

16. The method of claim 1, wherein the step of moderately heating the biological spores comprises an external heat source.

17. The method of claim 1, wherein the biological microbes comprises endospores.

18. The method of claim 1, wherein the biological microbes comprises Bacillus endospores.

19. The large-scale decontaminated microbe product produced by the method of claim 1.

20. A method for large-scale decontamination of biological microbes, comprising the steps of:
washing a biological microbe contaminant from a decontamination site into a waste water product;
applying an acidic environment to the contaminated wash product; and,
applying an amine oxide compound in combination with the acidic environment to the wash product effective for decontamination of the biological microbes within the wash product.
